(19) United States
(12) Patent Application Publication
(10) Pub. No.: US 2022/0390462 A1
(43) Pub. Date: Dec. 8, 2022

(54) METHOD FOR RAPID DETECTION OF DRY EYE SYNDROME

(71) Applicant: WENZHOU MEDICAL UNIVERSITY, Wenzhou (CN)

(72) Inventor: FEI LIU, Cupertino, CA (US)

(21) Appl. No.: 17/338,950

(22) Filed: Jun. 4, 2021

(51) Int. Cl. G01N 33/68; H01J 49/16; H01J 49/00

(52) U.S. Cl. CPC G01N 33/6842; G01N 33/6851; G01N 33/6893; H01J 49/164; H01J 49/0036; G01N 2800/16

(57) ABSTRACT

A method for rapid detection of dry eye syndrome includes collecting a first tear fluid from healthy participant and a second tear fluid from patient with eye dryness; isolating EV samples from the first tear fluid; acquiring a first fingerprint diagram of proteomes of the EV samples from the first tear fluid, the first fingerprint diagram comprises a plurality of first discriminant peaks; isolating EV samples from the second tear fluid; acquiring a second fingerprint diagram of proteomes of the EV samples from the second tear fluid, the second fingerprint diagram comprises a plurality of second discriminant peaks; and comparing the first discriminant peaks and the second discriminant peaks to determine whether the patient has the DES. This is a fast and precise method for detecting the DES of the participant.

### FIELD

The subject matter herein generally relates to biotechnology, and more particularly, to a method for rapid detection of dry eye syndrome.

### BACKGROUND

Dry eye syndrome (DES) is a common ocular surface disease characterized by tear film homeostasis disruption or neurosensory abnormalities. A fast and precise method for detecting the DES of the participant is needed.

### BRIEF DESCRIPTION OF THE DRAWINGS

Implementations of the present disclosure will now be described, by way of embodiments only, with reference to the attached figures.

FIG. 1 is a flowchart of an embodiment of a method for rapid detection of dry eye syndrome according to the present disclosure.

FIG. 2 is a sub-flowchart of block 12 of FIG. 1.

FIG. 3 is a sub-flowchart of block 13 of FIG. 1.

FIG. 4 is a sub-flowchart of block 16 of FIG. 1.

FIG. 5 is a flowchart of an embodiment of a method for acquiring the first fingerprint diagram of proteomes or the second fingerprint diagram of proteomes by MALDI-TOF-MS according to the present disclosure.

FIG. 6a is a diagram of the EVs molecule in the tear.

FIG. 6b is a morphology image of tear-EVs captured by TEM.

FIG. 6c is a diagram of size distributions of particles in tear-EVs and tear measured by dynamic light scattering.

FIG. 6d is western blot detection of the tear-EVs and the tear.

FIG. 6e shows silver staining of tear and tear-EVs.

FIG. 7a is mass spectra of the tear-EVs.

FIG. 7b is a diagram of the mean signal-to-noise (S/N) ratio of the tear-EVs. The S/N is obtained from eight peaks marked with asterisks in the range m/z 4000-18,000 in FIG. 6a.

FIG. 7c is mass spectra of the tear.

FIG. 7d is a diagram of the mean signal-to-noise (SIN) ratio of the tear. The S/N is obtained from six peaks marked with asterisks in the range m/z 4000-18,000 in FIG. 7c.

FIG. 8a is a virtual gel graph of mass spectra obtained from the tear and the tear-EVs.

FIG. 8b is mean peak illustration of tear-EVs (black line) and tear (grey line) dealt with Clin Pro Tools v 3.0.

FIG. 9a is an image of clustering analysis of mass spectra using the combination of two discriminant peaks (2878 and 2887 Da), the x-axis and y-axis representing the relative intensity of the tear at peaks of 2887 and 2878 Da, respectively.

FIG. 9b is a virtual gel graph of mass spectra in a range of 2700 Da to 3000 Da obtained from the tear and the tear-EVs.

FIG. 9c is mean peak illustration of differential peaks at 2886.99 Da, 2876.23 Da, and 2894.29 Da of tear-EVs (black line) and tear (grey line) dealt with Clin Pro Tools v 3.0.

FIG. 9d is a virtual gel graph of mass spectra in a range of 7000 Da to 8000 Da obtained from the tear and the tear-EVs.

FIG. 9e is mean peak illustration of differential peak at 7344.84 Da of tear-EVs(black line) and tear (grey line) dealt with Clin Pro Tools v 3.0.

FIG. 10a, FIG. 10b, and FIG. 10c are diagrams of four most discriminant peaks of tears derived from HC and DES groups.

FIG. 10d is an image of clustering analysis of mass spectra of the tear, the x-axis and y-axis representing the relative intensity of tear at 1957 Da and 1941 Da, respectively.

FIG. 10e is a diagram of mass spectra of tear-EVs from DES and HC.

FIG. 11 is a diagram of the relative area of peak in 17435 Da in DES and HC groups, applying Mann-Whitney U test, n=4, p=0.029.

### DETAILED DESCRIPTION

It will be appreciated that for simplicity and clarity of illustration, where appropriate, reference numerals have been repeated among the different figures to indicate corresponding or analogous components. In addition, numerous specific details are set forth to provide a thorough understanding of the embodiments described herein. However, it will be understood by those of ordinary skill in the art that the embodiments described herein can be practiced without these specific details. In other instances, methods, procedures, and components have not been described in detail so as not to obscure the related relevant feature being described. Also, the description is not to be considered as limiting the scope of the embodiments described herein. The drawings are not necessarily to scale and the proportions of certain parts may be exaggerated to better illustrate details and features of the present disclosure.

The term “comprising,” when utilized, means “including, but not necessarily limited to”; it specifically indicates open-ended inclusion or membership in the so-described combination, group, series, and the like.

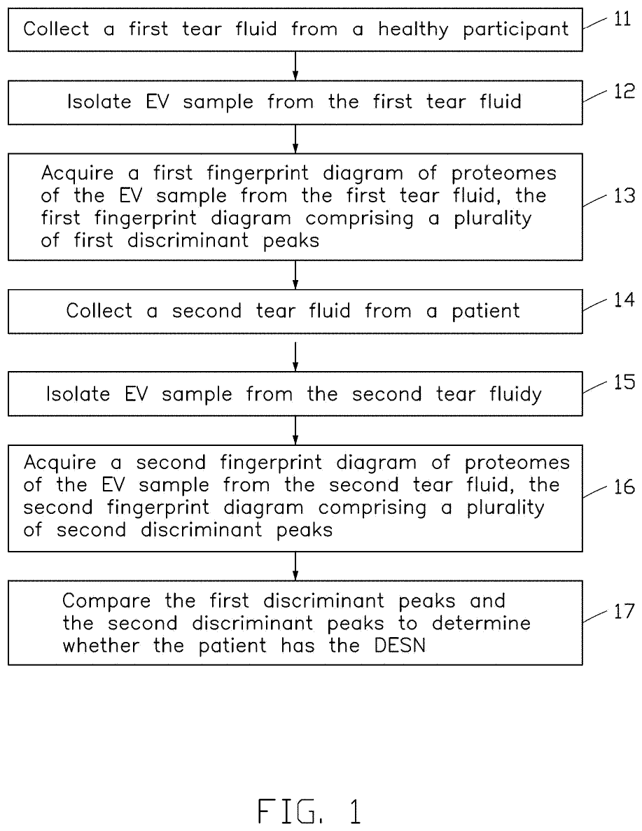

FIG. 1 illustrates an embodiment of a method for rapid detection of dry eye syndrome (DES). The method is provided by way of embodiment, as there are a variety of ways to carry out the method. The method can begin at block 11.

At block 11, fluid from the eye (first tear fluid) is collected from a healthy participant not suffering from DES.

In an embodiment, the first tear fluid is collected from both eyes by placing a Schirmer test strip (Jingming, Tianjin, China) on each eye of each healthy participant. The collection is done at room temperature, and the strip length is recorded after 5 min of collection and at completion. Schirmer strips from both eyes are then transferred to 5 mL of phosphate-buffered saline (PBS; Gibco, pH 7.4; Thermo Fisher Scientific, Oslo, Norway) and then stored at −80° C.

At block 12, extracellular vesicle (EV) sample is isolated from the first tear fluid.

After isolation, a concentrated and purified EV sample is collected for the downstream characterization. Particle concentration, particle size, morphology, and proteomes expression of the EV samples from the first tear fluid are characterized for quality control.

In an embodiment, the EV sample from the first tear fluid is analyzed by nanoparticle tracking using a Nano Sight NS 300 (Malvern, UK) to measure particle concentration of the EV sample.

In an embodiment, dynamic light scattering (DLS) is performed to measure the particle size of the EV sample on a Malvern Zetasizer Nano ZS ZEN 3600 (Malvern, UK). For each measurement, the EV sample is eluted to 300 μL and measured in triplicate.

In an embodiment, the particle size of the EV sample is in a range of 152.8±42.1 nm.

In an embodiment, the morphology of the EV sample is captured by Transmission Electron Microscopy (TEM).

In an embodiment, the morphology of the vesicles of the EV sample is spherical and cup shaped.

In an embodiment, the proteomes expression of the EV sample isolated from the first tear fluid is characterized by Silver Staining and Western Blot.

In an embodiment, specific EV markers including tetraspanin family proteins (CD 9 and CD 81) and EV trafficking-related proteins (Alix, TSG 101) are detected by western blot method.

At block 13, a first fingerprint diagram of proteomes of the EV sample from the first tear fluid is acquired, the first fingerprint diagram comprises a plurality of first discriminant peaks.

In an embodiment, the first fingerprint diagram of proteomes of the EV sample from the first tear fluid is acquired by Matrix-Assisted Laser Desorption Ionization/Time-of-Flight Mass Spectrometry (MALDI-TOF-MS).

At block 14, fluid from the eye of a patient complaining of eye dryness (second tear fluid) is collected.

In an embodiment, patients are diagnosed with DES if they reported dry eye symptoms and presented either of the following clinical examination results:(1) abnormalities in the Schirmer I test (≤5 mm/5 min) or tear breakup time (TBUT)≤5 s, and (2) indicative fluorescein staining of the corneal epithelium along with a TBUT between 5 and 10 s or a Schirmer I test result between 5 and 10 mm per 5 min.

In an embodiment, the second tear fluid is collected from both eyes of patient by placing a Schirmer test strip (Jingming, Tianjin, China) on each eye. The collection is done at room temperature, and the strip length is recorded after 5 min of collection and at completion. Schirmer strips from both eyes are then transferred to 5 mL of phosphate-buffered saline (PBS; Gibco, pH 7.4; Thermo Fisher Scientific, Oslo, Norway) and then stored at −80° C.

At block 15, EV sample is isolated from the second tear fluid.

In an embodiment, a method for isolating EV sample from the second tear fluid is same with the method for isolating EV sample from the first tear fluid.

At block 16, a second fingerprint diagram of proteomes of the EV sample from the second tear fluid is acquired, the second fingerprint diagram comprises a plurality of second discriminant peaks.

In an embodiment, the first fingerprint diagram of proteomes of the EV sample from the first tear fluid is acquired by Matrix-Assisted Laser Desorption ionization/Time-of-Flight Mass Spectrometry (MALDI-TOF-MS).

At block 17, the first discriminant peaks and the second discriminant peaks are compared to determine whether the patient has the DES.

In an embodiment, the second fingerprint diagram comprises four second discriminant peaks. Intensities of the four second discriminant peaks are weaker in the first fingerprint diagram than in the second fingerprint diagram. Mass-to-charge ratios of the four second discriminant peaks are in ranges of 2100-2200 Da, 2700-2800 Da, and 1900-2000 Da.

In an embodiment, mass-to-charge ratios of the four second discriminant peaks comprise 2158.12 Da, 2740.41 Da, 1940.77 Da, and 1957.14 Da.

In an embodiment, the first fingerprint diagram comprises one first discriminant peak. An intensity of the first discriminant peak is weaker in the second fingerprint diagram than in the first fingerprint diagram. Mass-to-charge ratio of the first discriminant peak is in a range of 16000-18000 Da.

In an embodiment, mass-to-charge ratio of the first discriminant peak comprises 17435 Da.

In an embodiment, the method for detecting DES can further be used to detect a wide range of diseases besides ocular abnormity, such as central nervous system disease, breast cancer, and Sjogren's syndrome.

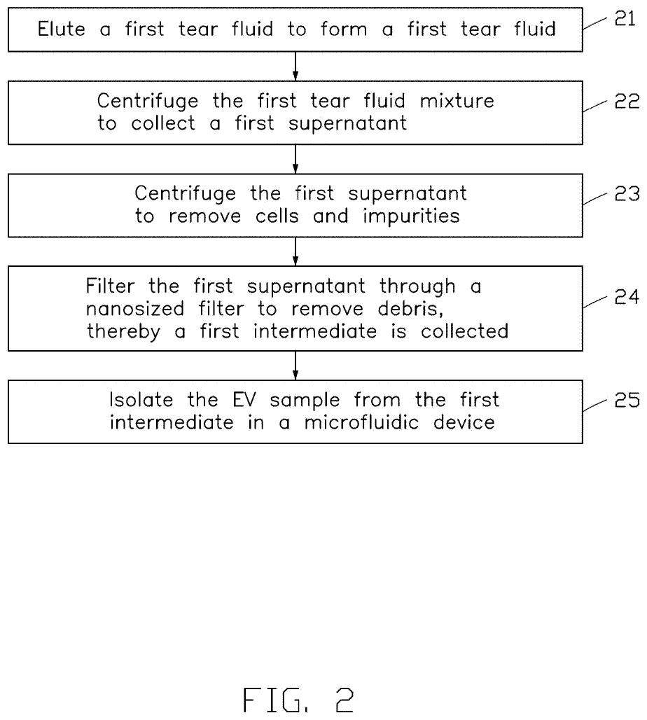

Referring to FIG. 2, block 12 can be carried out as follows.

At block 21, the first tear fluid is eluted from Schirmer strips into PBS to form a first tear fluid mixture.

At block 22, the first tear fluid mixture is centrifuged to collect a first supernatant.

In an embodiment, the first tear fluid mixture is centrifuged at 200 g for 10 min.

At block 23, the first supernatant is centrifuged to remove cells and impurities.

In an embodiment, the first supernatant is centrifuged at 3000 g for 10 min to remove cells and impurities.

At block 24, the first supernatant is filtered through a nanosized filter to remove debris, thereby a first intermediate sample is collected.

In an embodiment, a size of the nanosized filter is 0.10 μm-0.25 μm.

In an embodiment, the size of the nanosized filter is 0.22 μm.

At block 25, the EV sample from the first intermediate sample is isolated in a microfluidic device.

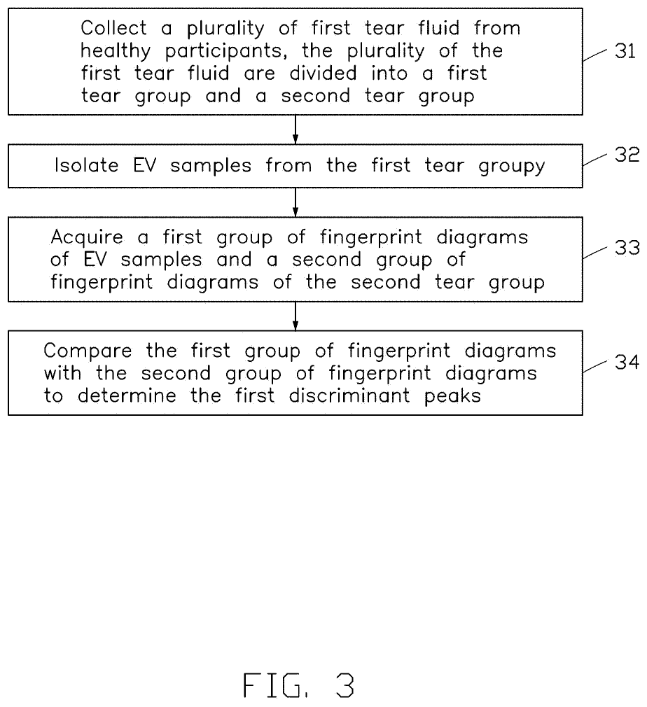

Referring to FIG. 3, block 13 can be carried out as follows.

At block 31, a plurality of first tear fluids is collected from healthy participants, the plurality of the first tear fluids is divided into a first tear group and a second tear group.

At block 32, EV samples are isolated from the first tear group.

At block 33, a first group of fingerprint diagrams of EV samples and a second group of fingerprint diagrams of the second tear group are acquired.

At block 34, the first group of fingerprint diagrams with the second group of fingerprint diagrams are compared to determine the first discriminant peaks.

In an embodiment, the first discriminant peaks are determined using a built-in mathematical model's genetic algorithm (GA) and a neural network algorithm.

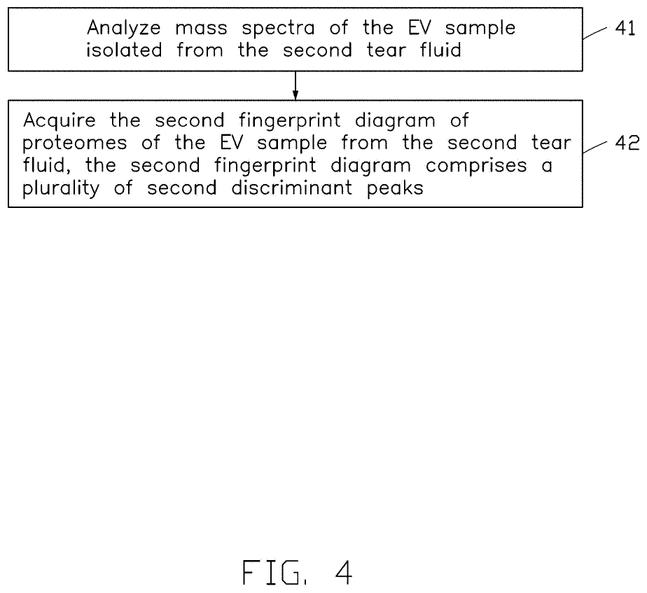

Referring to FIG. 4, block 16 can be carried out as follows.

At block 41, mass spectra of the EV sample isolated from the second tear fluid is analyzed.

At block 42, the second fingerprint diagram of proteomes of the EV sample from the second tear fluid is acquired, the second fingerprint diagram comprises a plurality of second discriminant peaks.

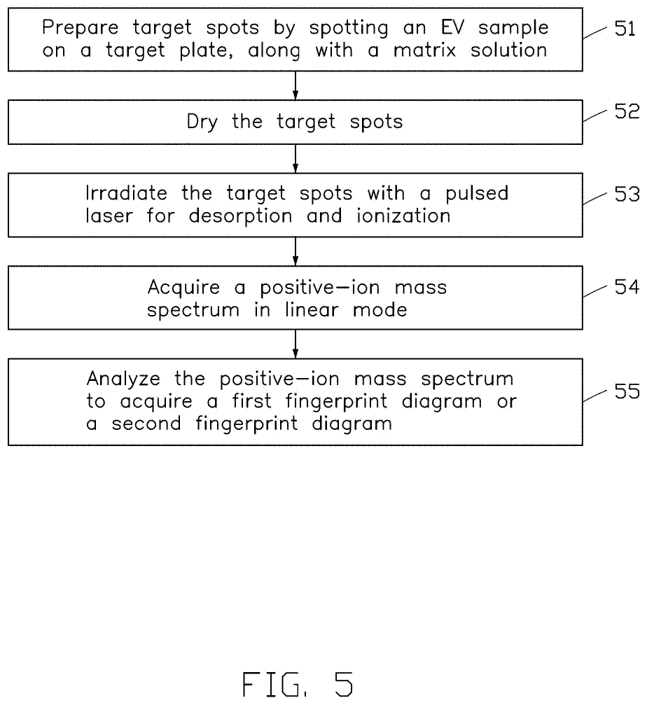

Referring to FIG. 5 illustrates an embodiment of a method for acquiring the first fingerprint diagram of proteomes of the EV sample from the first tear fluid or the second fingerprint diagram of proteomes of the EV sample from the second tear fluid by MALDI-TOF-MS. The method is provided by way of embodiment, as there are a variety of ways to carry out the method. The method can begin at block 51.

At block 51, target spots are prepared by spotting the EV sample on a target plate, along with a matrix solution.

In an embodiment, the matrix solution comprises alphacyano-4-hydroxycinnamic acid matrix.

At block 52, the target spots are dried.

At block 53, the target spots are irradiated with a pulsed laser for desorption and ionization.

In an embodiment, a wavenumber of the pulsed laser is in a range of 300 nm-400 nm, a time of irradiating the target spots with the pulsed laser is in a range of 100 ns-150 ns.

At block 54, a positive-ion mass spectrum is acquired in linear mode.

In an embodiment, each target spot is irradiated to desorb five thousand shots. Five thousand shots are averaged to obtain the positive-ion mass spectrum.

At block 55, the positive-ion mass spectrum is analyzed to acquire the first fingerprint diagram or the second fingerprint diagram.

### EXAMPLE

The first tear fluid was collected from a healthy participant. The first tear fluid was collected from both eyes by placing a Schirmer test strip (Jingming, Tianjin, China) on each eye of each healthy participant. The collection was done at room temperature, and the strip length was recorded after 5 min of collection and at completion. Schirmer strips from both eyes were then transferred to 5 mL of phosphate-buffered saline (PBS; Gibco, pH 7.4; Thermo Fisher Scientific, Oslo, Norway) and then stored at −80° C.

The second tear fluid was collected from a patient.

Patients were diagnosed with possible DES if they reported dry eye symptoms and presented either of the following clinical examination results: (1) abnormalities in the Schirmer I test (≤5 mm/5 min) or tear breakup time (TBUT)≤5 s and (2) indicative fluorescein staining of the corneal epithelium along with a TBUT between 5 and 10 s or a Schirmer I test result between 5 and 10 mm per 5 min. The second tear fluid was collected from both eyes by placing a Schirmer test strip (Jingming, Tianjin, China) on each eye of each patient. The collection was done at room temperature, and the strip length was recorded after 5 min of collection and at completion. Schirmer strips from both eyes were then transferred to 5 mL of phosphate-buffered saline (PBS; Gibco, pH 7.4; Thermo Fisher Scientific, Oslo, Norway) and then stored at −80° C.

EV samples were isolated from the first tear fluid and the second fluid. EV samples were isolated by a nanosized filter-based method. This method, utilizing size-based filtration, was especially suitable for the samples with limited volume such as tears. For each sample, the first tear fluid or the second fluid was eluted from Schirmer strips into PBS (5 mL; pooling of 2.5 mL PBS containing a 30 mm Schirmer strip from each eye) and was centrifuged at 200 g for 10 min. The supernatants were collected and centrifuged at 3000 g for 10 min to remove cells and impurities. The supernatants were filtered through a 0.22 μm filter to remove debris to collect intermediate samples. The intermediate samples were used in the microfluidic device to isolate the EV samples. After isolation, each concentrated and purified EV sample was collected for the downstream characterization and mass spectra analysis.

A particle concentration and a particle size of the EV sample from the first tear fluid and the first tear fluid (tear sample) were analyzed by Nanoparticle Tracking Analysis and Dynamic Light Scattering, respectively.

The EV samples and the tear samples were analyzed by nanoparticle tracking using a Nano Sight NS 300 (Malvern, UK) to measure the concentration of the particles. EV samples from the first tear fluid were then diluted using PBS and infused manually into the machine, with six videos of 30 s each. The analysis was done using Nanoparticle Tracking Analysis software (Version 3.4, Build 3.4.003), and each sample was tracked at a camera level of 16, with a detection threshold set at 5. Approximately 20-80 particles could be observed m the field of view. Dynamic light scattering (DLS) was performed to measure the particle size of the EV samples and the tear samples on a Malvern Zeta sizer Nano ZS ZEN 3600 (Malvern, UK). For each measurement, the EV sample from the first tear fluid was eluted to 300 μL and measured in triplicate.

The proteomes expression of the EV sample isolated from the first tear fluid was characterized by Silver Staining and Western Blot. The protein concentrations of the EV samples from the first tear fluid and the tear sample were measured by Qubit assay (Invitrogen, USA). The EV samples from the first tear fluid and the tear sample were loaded on two separate SDS-PAGE gels (Express Plus TM PAGE Gels; Genescript, USA). One gel was used for silver staining with the Fast Silver Stain Kit (Sangon Biotech, China) according to the manufacturer's instructions, whereas the other was used in, the western blot method. The latter gel was transferred to a polyvinylidene difluoride membrane before being blocked for 1 h in 5% nonfat dry milk. Primary antibodies used for immunoblotting include rabbit monoclonal antihuman TSG 101 (1:1000; Abeam, USA), rabbit monoclonal antihuman CD 9 (1:1000; Cell Signaling Technology, USA), mouse monoclonal antihuman CD 81(1:1000; Santa Cruz, USA), mouse monoclonal antihuman Alix (1:1000; Santa Cruz, USA), mouse monoclonal antihuman Mac-2 BP (1:1000; Santa Cruz, USA), and mouse monoclonal antihuman albumin (1:1000; Santa Cruz, USA). For the chemiluminescent detection of proteins, antimouse or antirabbit IgG-HRP secondary antibodies (1:3000; Santa Cruz, USA) and HRP chemiluminescent substrates (Super Signal TM West Pico PLUS Chemiluminescent Substrate; Thermo Fisher Scientific) were used, and relative protein levels were determined using Image J software (v 1.8.0; Germany).

The morphology of the EV sample from the first tear fluid was captured by Transmission Electron Microscopy (TEM). The EV sample from the first tear fluid was mixed with an equal volume of 4% paraformaldehyde and deposited on formvar carbon-coated grids (Plano, Wetzlar, Germany) by floating the grid on 20 μL of the mixed sample for 20 min. After removing any excess liquid by a filter paper, negative staining was performed with 2% uranyl acetate for 30 s at room temperature. Images were then captured using transmission electron microscopy (TEM; Helios Nano lab Dual Beam, FET, USA) at 80 kV

The first fingerprint diagram of proteomes of the EV sample from the first tear fluid and the tear sample were acquired by MALDI-TOF-MS.

MALDI-TOF-MS targets were prepared by spotting 0.5 μL of each purified sample on a Ground Steel Chip 600/384 IF plate, along with a 0.8 μL drop of MALDI matrix solution (alphacyano-4-hydroxycinnamic acid matrix [0.3 mg/mL in ethanol: acetone (2:1) solution]). After drying, the EV sample and the tear sample were analyzed using an Auto flex max TOF/TOF with Flex Control 3.0 (Bruker Daltonics, Autoflex, Leipzig, Germany) operated in linear mode geometry. Sample spots were irradiated with a pulsed laser (360 nm, 120 ns) for desorption and ionization. Five thousand shots were fired per spot and averaged to obtain representative mass spectra. Positive-ion mass spectra acquired in linear mode were collected at an acceleration voltage of 20 kV, with the calibration of the MALDI mass spectra performed using Protein Calibration Standard I (Barker Daltonics) as standards. The built-in mathematical model's genetic algorithm (GA) and neural network algorithm were used to select each peptide peak, and classification models were set up using Clin Pro Tools 3.0 software to determine the optimal separation planes between samples from the two training groups. The mass spectromehy proteomics data were deposited in the Proteome Xchange Consortium via the PRIDE 29 partner repository with the dataset identifier PXD 020217.

Characterization of the EV sample from the first tear fluid and the tear sample were analyzed by MALDI-TOF-MS.

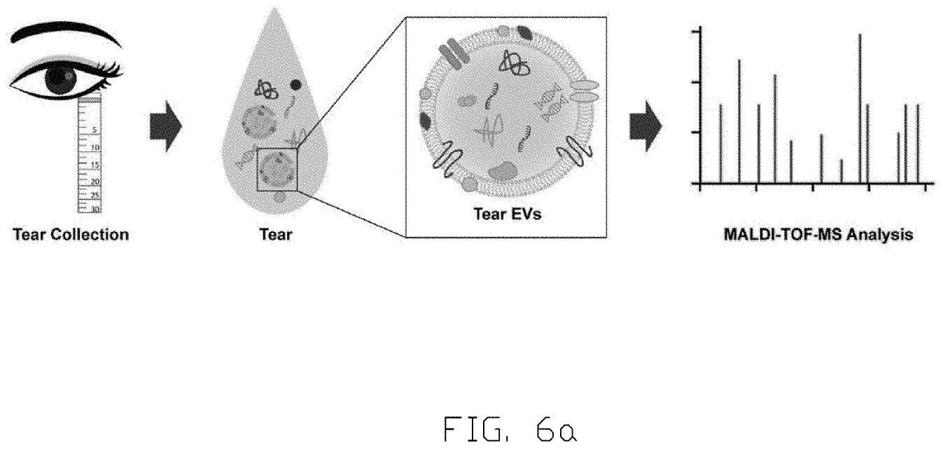

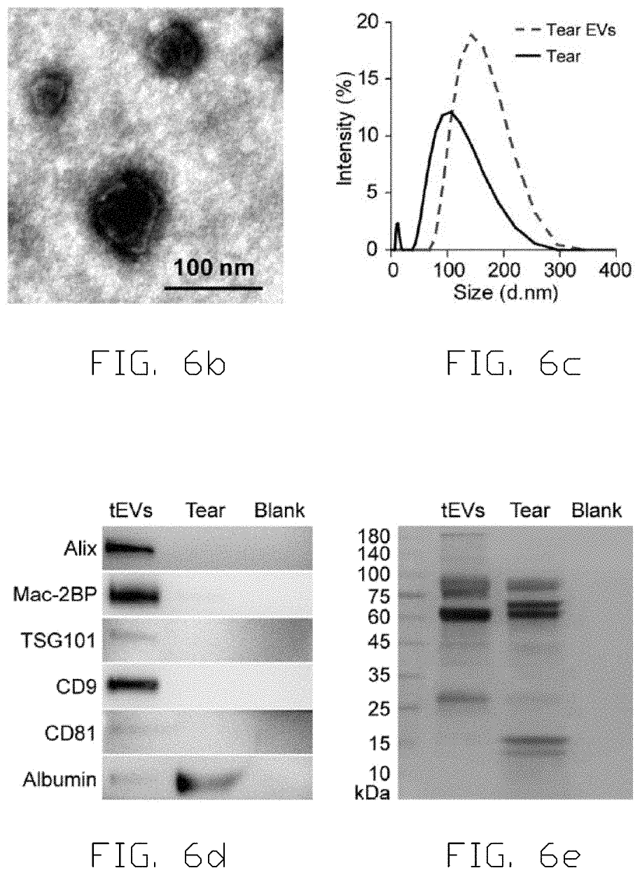

Schirmer test strips were used to collect tears from subjects because of its relatively large tear volume. The EV sample from the first tear fluid (tear-EVs) and the tear sample (tear) were analyzed by MALDI-TOF-MS to acquire the fingerprint of proteomes (FIG. 6a). For quality control, morphology, size, and protein expression from tear-EVs were characterized. The morphology of the tear-EVs and the tear was captured by TEM (FIG. 6b), and the spherical and cup-shaped vesicles were observed. Besides, the EVs were an important constitution of tear demonstrated in this study. To better understand their biophysical features, the size distribution of tear and tear-EVs were analyzed. DLS results showed that the EV sample had a wide range of sizes (152.8±42.1 nm) (FIG. 6c). By comparing the particle size contained in the tear and the tear-EVs, larger particle size was seen in the tear-EVs. This shift may result from EV enrichment during the isolation process. The specific EV markers including tetraspanin family proteins (CD 9 and CD 81) and EV trafficking-related proteins (Alix, TSG 101) were detected by western blot method. These markers were all detected in the tear-EVs but not in the tear. Conversely, albumin, which was a high abundance protein in tears from plasma leakage, can only be detected in the tear samples, indicating the good performance of EV purification (FIG. 6d). Not all the markers were detected in the blank control, demonstrating that the protein contamination caused by the Schirmer strip was limited. Moreover, the total protein expression pattern was visualized by silver staining, and the result also showed the changes in the protein profile during EV enrichment and purification (FIG. 6e).

Characterization of the tear-EVs was analyzed by MALDI Mass Spectra.

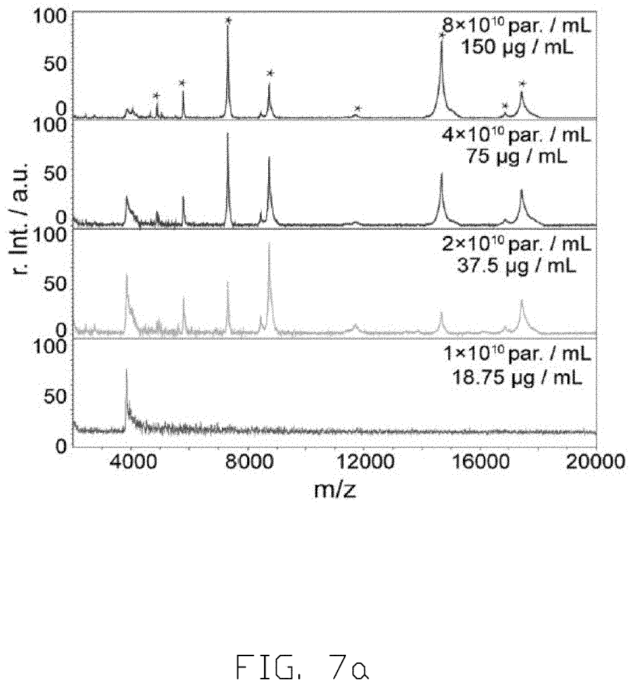

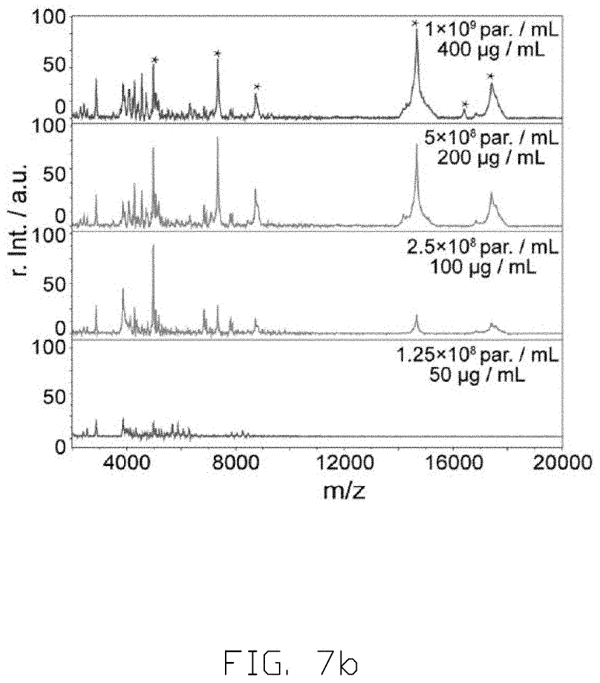

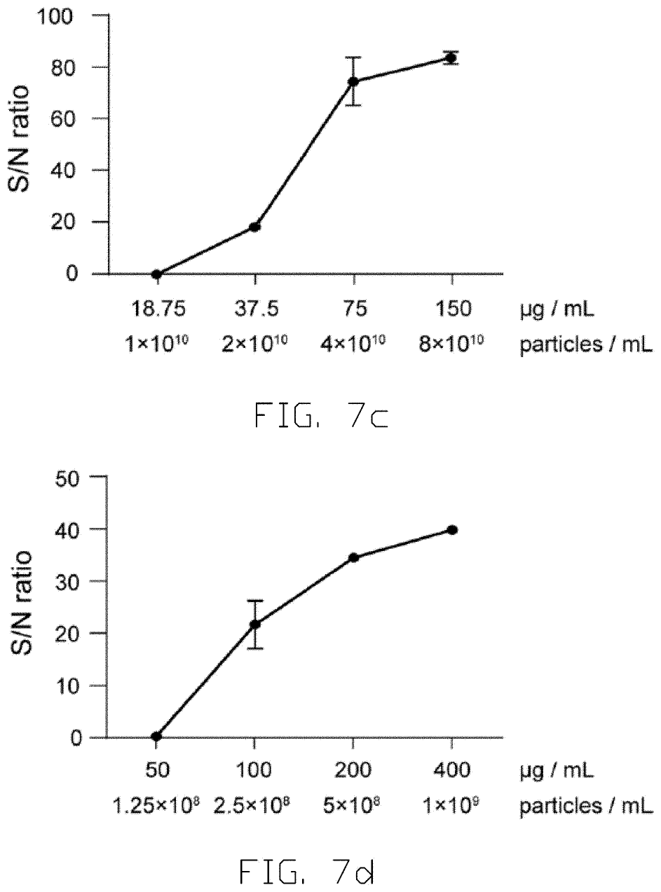

The MALDI detection of the tear-EVs and the tear was in demand. To test the limit of detection (LOD) for MALDI-TOF-MS, the tear-EVs and the tear with increased levels were first evaluated. In the tear-EVs model, eight most abundant peaks (m/z 5791±1, 7344±1, 7411±1, 8724±2, 14.683±6, 14,973±23, 17,440±24, and 17,617±1) were considered for averaging, their S/N ratios (FIG. 7a, FIG. 7b). The mass spectra obtained were informative beyond 1.0×1010 particles/mL (≈18.75 μg/mL) (FIG. 7b). As reported, a high-quality mass spectrum can be obtained from exosomes or EVs with a concentration of 5×1010 particles/mL isolated from cell culture. The result corresponds to the EV amounts found in the cell culture and human plasma reported in other studies. Moreover, the LOD of protein and particle concentration in the tear model was evaluated. As shown in FIG. 7c and FIG. 7d, the average of six most abundant peaks is considered (m/z 4974±1, 7345±2, 8727±2, 14,675±3, 17,421±7, and 17,582±21). The mass spectra were informative down to 1.25×108 particles/ML (≈50 μg/mL). In the case of comparable protein levels, the particle concentration required for the tear sample detection was lower than that for the tear-EVs. This is believed to be due to the abundant free proteins besides EVs present in tears. These tear proteins can provide fingerprinting information with stronger signals than EVs, further suggesting that tear-EVs can be very well purified in the studies.

Discriminant Mass Spectra of the tear-EVs and the tear were acquired.

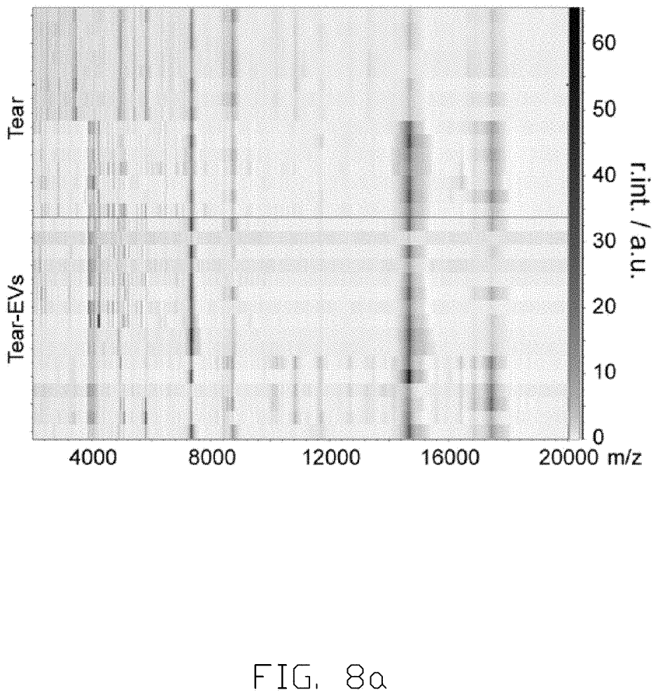

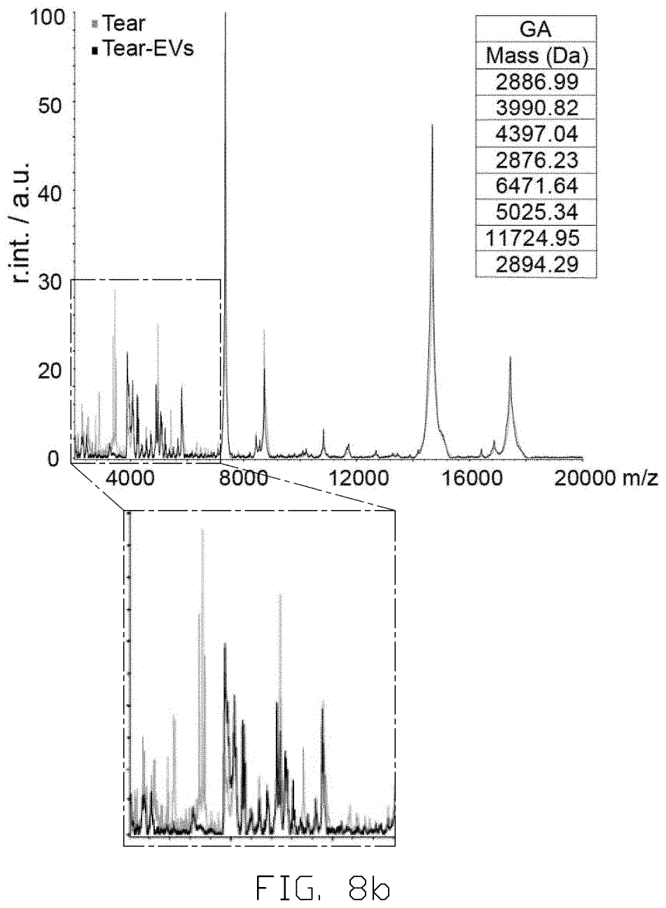

Tears were collected from 15 healthy individuals and then analyzed for the mass spectra of the tear and isolated EVs. After acquiring the fingerprints, discriminant peaks between the EV samples and the tear samples were compared. The virtual gel graph of mass spectra based on relative expression by Clin Pro Tools v 3.0 is shown in FIG. 8a. The mass spectra are obtained with the mass-to-charge ratio (m/z) between 20 and 20,000 Da. The average spectra of the tear sample (green line) and the EV sample (red line) are shown in FIG. 8b. All spectra are normalized to their total ion count before being used in model generation with the GA. This is necessary to make different groups comparable to each other. The eight discriminant peaks are listed in FIG. 8b (m/z: 2886.99, 3990.82, 4397.04, 2876.23, 6471.64, 5025.34, 11724.95, and 2894.29) with a cross-validation of 95.1%. In further exploration of the feasibility and accuracy of this signature peaks model, the recognition capability of tear and tear-EVs from patients with DES was also verified. The results showed that the sensitivity and specificity were 100% (4/4) and 91.7% (11/12), respectively, discriminating the tear-EVs from tear samples (treating the tear-EVs class as positive) (see Table 1). Thus, EVs specific fingerprints can be produced in both healthy and disease groups, demonstrating that MALDI-TOF-MS is a suitable method for tear-EV detection.

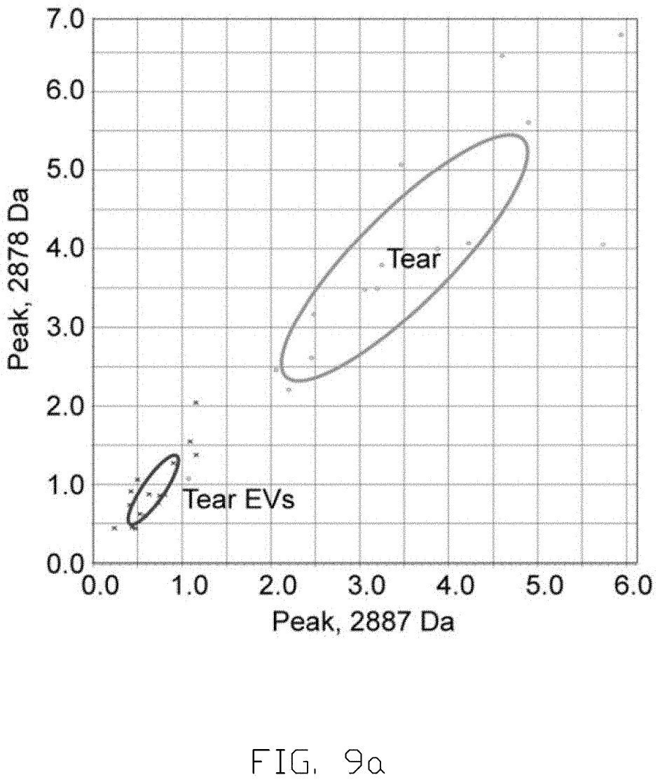

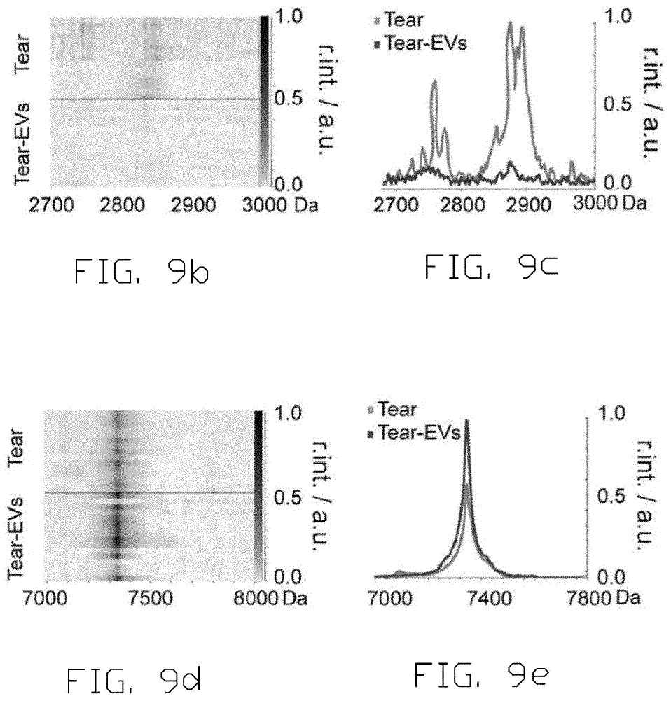

To investigate the changes in peptidome and proteome profiles during the EV isolation process, all the discriminant peaks (p<0.05) are listed (see Table 2). As shown in FIG. 9a, the peaks of m/z 2878 and 2887 distinguish EV from tears. Some of the discriminant peaks including 2886.99, 2876.23, 2894.29, and 7344.84 are illustrated by gel view and spectra view (FIG. 9b, FIG. 9c, FIG. 9d, and FIG. 9e). It is noticed that the intensities of most of these peaks decreased after EV isolation (FIG. 9b, FIG. 9c, and see Table 2), likely because of the effective removal of free peptides and proteins with small molecular weight during the purification procedure. Some peaks with increased intensity are also found in tear-EVs (4119.96, 3898.09, 3927.59, 3938.3, 3879.41, 7344.84, and 3990.82 Da). For example, the peak of 7344.84, which has a relative signal intensity higher than that of other peaks in both tear and EV samples (FIG. 9d, FIG. 9e, and Table 2). This peak indicates a high abundance of protein in tears. The peak in 7332 Da (theoretical mass: 7350 Da) of tear detected by MALDI-MS is proposed to be lysozyme 2 H+(LYZ). Lysozyme is well known as the antibacterial protein in tears and can be regarded as a diagnostic indicator because of the decreased expression in tears of DES patients. Additionally, LYZ is suggested to be involved in the EV-related biological process. In this study, the LYZ protein also showed high enrichment in tear-EVs, indicating a potential role of LYZ in the tear-EV function.

TABLE 1
the validation of the tear-EVs model by samples from DES patients,
4 samples of tear-EVs and 12 samples of tear being enrolled.

| | | Correct Classified Part | | | | |
|---|---|---|---|---|---|---|
| Class | Name | of Valid Spectra | 1 | 2 | 0 | Inv. |
| 1 | Tear EVs | 100% | 4 | 9 | 0 | 1 |
| 2 | Tear | 91.7% | 1 | 11 | 0 | 0 |

TABLE 2
differential peaks analyzed by Clin Pro Tools.

| mass (Da) | p value | mean 1 ± SD 1 | mean 2 ± SD 2 |
|---|---|---|---|
| 2886.99 | <0.001 | 0.75 ± 0.31 | 3.89 ± 1.54 |
| 2876.23 | <0.001 | 1.03 ± 0.49 | 4.31 ± 1.74 |
| 2894.29 | <0.001 | 0.78 ± 0.39 | 4.59 ± 2.08 |
| 6344.15 | 0.0346 | 0.6 ± 0.34 | 1.64 ± 0.94 |
| 4978.68 | 0.0597 | 2.59 ± 1.61 | 6.18 ± 3.45 |
| 4119.96 | 0.0105 | 3.11 ± 1.23 | 1.71 ± 0.91 |
| 5025.34 | 0.0231 | 0.86 ± 0.57 | 1.88 ± 0.94 |
| 2482.09 | 0.0238 | 1.1 ± 0.74 | 3.01 ± 1.93 |
| 3898.09 | 0.0331 | 7.23 ± 4.4 | 3.18 ± 2.45 |
| 3927.59 | 0.0194 | 6.56 ± 3.62 | 3.11 ± 1.96 |
| 3938.3 | 0.0193 | 7.35 ± 5.14 | 3.04 ± 2.24 |
| 3879.41 | 0.0438 | 9.12 ± 5.51 | 4.39 ± 3.48 |
| 7344.84 | 0.0417 | 32.82 ± 17.65 | 19.72 ± 6.1 |
| 3990.82 | 0.0417 | 3.72 ± 2.06 | 8.06 ± 10.65 |
| 3487 | 0.0167 | 0.6 ± 0.33 | 8.06 ± 10.65 |
| 2272.33 | 0.0416 | 2.22 ± 2.2 | 3.87 ± 1.85 |

The p value was calculated by Student's t-test if data obeys the normal distribution or calculated by the Wilcoxon test. The mean is the average intensity. The SD is the standard deviation of relative intensity. The class 1 is tear-EVs, and the class 2 is tear.

Identification of Tear and Tear-EVs biomarkers for classifying DES.

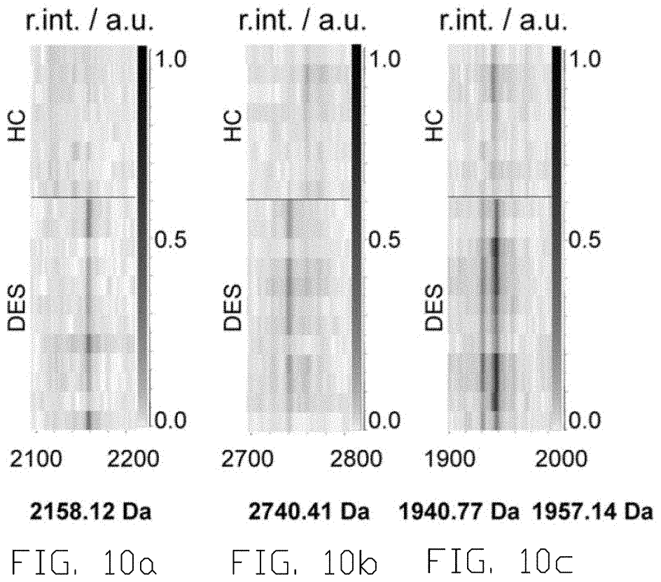

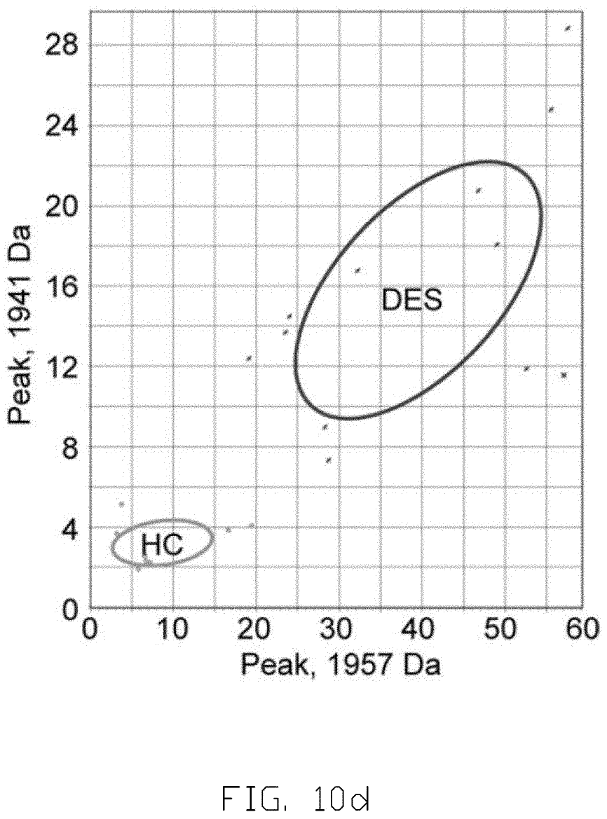

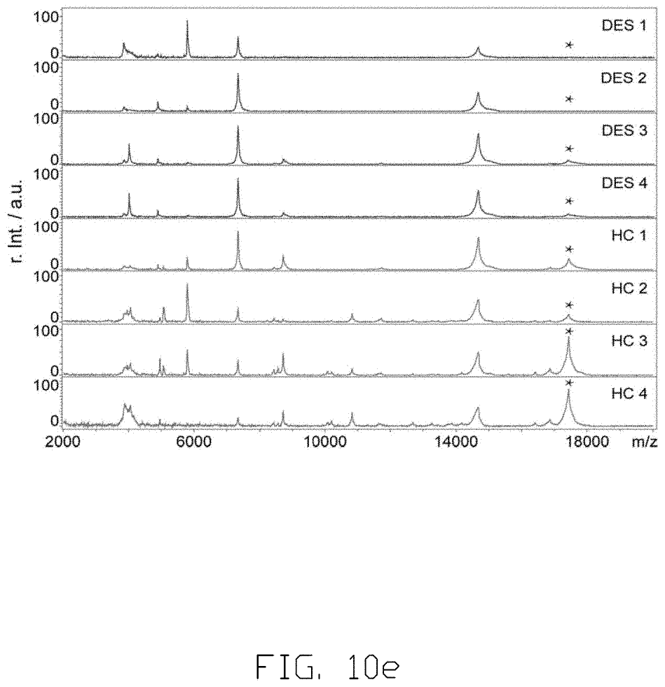

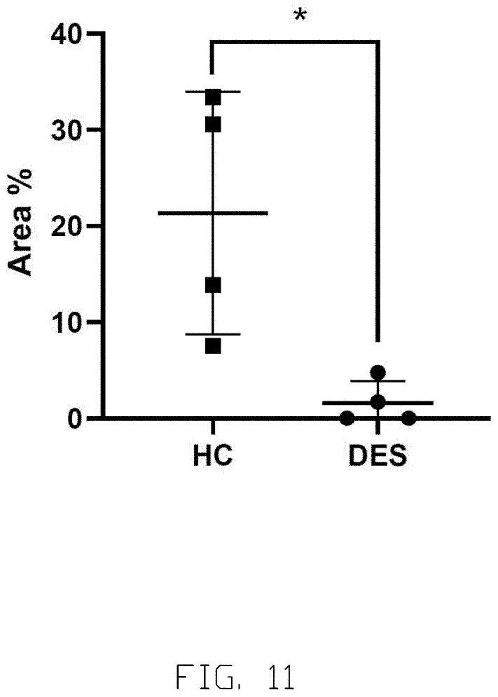

This study reveals the feasibility of the MALDI-TOF-MS application in the tear-based DES diagnosis. The clinical information used in DES studies include age and the OSDI score of six DES patients and four Healthy Control (HC) donors (Table 3), and all the discriminant peaks were listed in Table 4. Tear samples from patients and healthy donors are first analyzed. The gel view of the four most discriminant peaks including 2158.12, 2740.41, 1940.77, and 1957.14 Da (p<0.01) are shown in FIG. 10a, FIG. 10b, and FIG. 10c, respectively. The results of two-dimensional peak distribution demonstrate that the peaks in 1957 and 1940.77 Da (correspond to 1941 Da) have an obvious discriminatory ability to distinguish DES from the BC group (FIG. 10d). Tear-EVs mass spectra can be acquired by MALDI-MS, and its performance is investigated in distinguishing DES from the TIC group. The mass spectra from four patients and four healthy participants are shown in FIG. 10e. Peaks at 17435 Da are marked with an asterisk (FIG. 10e), and the relative area of m/z shows a significant decrease in the DES group (FIG. 11). By searching the protein in UniProt (https://www.uniprot.org) based on molecular weight and literature research, this peak is inferred as lipocalin, which was present a down regulated expression in a DES patient's tear. The abovementioned results reveal the potential of MALDI-TOF-MS fingerprinting technology for rapidly identifying the tear and EV-based biomarker of ocular disease of patients from healthy controls.

TABLE 3
clinical Information of DES patients and HC donors
included in the tear-based dry eye classification.

| group | N | age | OSDI |
|---|---|---|---|
| DES | 6 | 40.14 ± 12.58 | 20.00 ± 11.02 |
| HC | 4 | 26.5 ± 0.5 | 5.95 6.04 |

The OSDI is Ocular Surface Disease Index questionnaire score.

TABLE 4
differential peaks of tear samples from DES and HC (p < 0.05).

| mass (Da) | p value | mean 1 ± SD 1 | mean 2 ± SD 2 |
|---|---|---|---|
| 1957.14 | <0.001 | 43.93 ± 16.44 | 9.62 ± 6.67 |
| 1940.77 | <0.001 | 17.56 ± 7.12 | 3.55 ± 1.27 |
| 2740.41 | <0.001 | 12.03 ± 5.29 | 2.28 ± 0.46 |
| 2158.12 | <0.001 | 15.11 ± 8 | 3.01 ± 1.12 |
| 2502.86 | 0.00281 | 2.1 ± 0.41 | 5.17 ± 1.31 |
| 5106.11 | 0.00281 | 1.53 ± 0.91 | 3.12 ± 0.56 |
| 8564.06 | 0.00281 | 1.49 ± 0.36 | 2.57 ± 0.5 |
| 6174.13 | 0.00281 | 0.8 ± 0.19 | 1.26 ± 0.22 |
| 14690.42 | 0.00974 | 6.44 ± 1.66 | 4.02 ± 1.11 |
| 4981.3 | 0.00974 | 6 ± 1.19 | 8.35 ± 1.32 |
| 5938.08 | 0.0146 | 0.85 ± 0.37 | 1.66 ± 0.5 |
| 5864.31 | 0.0194 | 3.37 ± 1.23 | 4.92 ± 0.76 |
| 8609.64 | 0.0194 | 1.21 ± 0.35 | 1.95 ± 0.48 |
| 8517.62 | 0.0223 | 0.97 ± 0.31 | 1.64 ± 0.46 |
| 13458.31 | 0.0223 | 0.98 ± 0.33 | 0.63 ± 0.13 |
| 2651.92 | 0.0362 | 7.5 ± 5.04 | 2.7 ± 0.72 |
| 4283.82 | 0.0362 | 1.6 ± 0.66 | 2.5 ± 0.62 |
| 5045.8 | 0.0467 | 1.83 ± 0.76 | 2.56 ± 0.34 |

The p-value was calculated by Student's t-test if the data obeys the normal distribution or can be calculated by the Wilcoxon test. The mean is the average intensity. The SD is the standard deviation of relative intensity. The class 1 is DES, and the class 2 is HC.

In summary, EV samples emerge as a useful tool in liquid biopsy. The efficient validation of EV quality and quantity lays a foundation for further molecular analysis and functional research. In this application, the feasibility of directly detecting intact tear-EVs as well as tears using MALDI-TOF-MS and is verified as concluding the LOD of the tear and tear-EV. By fingerprinting the characteristic peaks, significantly different expression profiles of peptides and low-molecular-weight proteins are seen when comparing the tear-EV and tear. The results of mass spectra of tear and tear-EVs from both DES and HC groups further validate a potential application in rapid DES diagnosis. This is a fast and precise method for detecting the DES of the participant. The detection of EVs diagnoses diseases more stably and accurately because of their ability to protect their contents. This method can also detect a wide range of diseases besides ocular abnormities. For example, tear-EVs have been reported to have a close relation with systemic diseases such as central nervous system disease, breast cancer, and Sjogren's syndrome.

The embodiments shown and described above are only examples. Therefore, many commonly known features and details are neither shown nor described. Even though numerous characteristics and advantages of the present technology have been set forth in the foregoing description, together with details of the structure and function of the present disclosure, the disclosure is illustrative only, and changes may be made in the detail, including in matters of shape, size, and arrangement of the parts within the principles of the present disclosure, up to and including the full extent established by the broad general meaning of the terms used in the claims. It will, therefore, be appreciated that the embodiments described above may be modified within the scope of the claims.

What is claimed is:

1. A method for rapid detection of dry eye syndrome (DES), comprising:
collecting a first tear fluid from a healthy participant;
isolating Extracellular vesicle (EV) sample from the first tear fluid;
acquiring a first fingerprint diagram of proteomes of the EV sample from the first tear fluid, the first fingerprint diagram comprising a plurality of first discriminant peaks;
collecting a second tear fluid from a patient;
isolating EV sample from the second tear fluid;
acquiring a second fingerprint diagram of proteomes of the EV sample from the second tear fluid, the second fingerprint diagram comprising a plurality of second discriminant peaks; and
comparing the first discriminant peaks and the second discriminant peaks to determine whether the patient has the DES.

2. The method of claim 1, wherein the first fingerprint diagram of proteomes and the second fingerprint diagram of proteomes are acquired by Matrix-Assisted Laser Desorption Ionization/Time-of-Flight Mass Spectrometry (MALDI-TOF-MS).

3. The method of claim 2, wherein the second fingerprint diagram comprises four second discriminant peaks, intensities of the four second discriminant peaks are weaker in the first fingerprint diagram than in the second fingerprint diagram, mass-to-charge ratios of the four second discriminant peaks are in ranges of 2100-2200 Da, 2700-2800 Da, and 1900-2000 Da.

4. The method of claim 3, wherein mass-to-charge ratios of the four second discriminant peaks comprise 2158.12 Da, 2740.41 Da, 1940.77 Da, and 1957.14 Da.

5. The method of claim 2, wherein the first fingerprint diagram comprises one first discriminant peak, an intensity of the first discriminant peak is weaker in the second fingerprint diagram than in the first fingerprint diagram, mass-to-charge ratio of the first discriminant peak is in a range of 16000-18000 Da.

6. The method of claim 5, wherein mass-to-charge ratio of the first discriminant peak comprises 17435 Da.

7. The method of claim 2, wherein acquiring the first fingerprint diagram of proteomes or the second fingerprint diagram of proteomes by MALDI-TOF-MS comprises:
preparing target spots by spotting the EV sample on a target plate, along with a matrix solution;
drying the target spots;
irradiating the target spots with a pulsed laser for desorption and ionization;
acquiring a positive-ion mass spectrum in linear mode;
analyzing the positive-ion mass spectrum to acquire the first fingerprint diagram or the second fingerprint diagram.

8. The method of claim 7, wherein the matrix solution comprises alphacyano-4-hydroxycinnamic acid matrix.

9. The method of claim 7, wherein a wavenumber of the pulsed laser is in a range of 300 nm-400 nm, a time of irradiating the target spots with the pulsed laser is in a range of 100 ns-150 ns.

10. The method of claim 7, wherein each target spot is irradiated to desorb five thousand shots, five thousand shots are averaged to obtain the positive-ion mass spectrum.

11. The method of claim 1, wherein acquiring the first fingerprint diagram of proteomes of the EV samples from the first tear fluid comprise:
collecting a plurality of first tear fluids from healthy participants, the plurality of the first tear fluids is divided into a first tear group and a second tear group;
isolating the EV samples from the first tear group;
acquiring a first group of fingerprint diagrams of EV samples and a second group of fingerprint diagrams of the second tear group; and
comparing the first group of fingerprint diagrams with the second group of fingerprint diagrams to determine the first discriminant peaks.

12. The method of claim 11, wherein the first discriminant peaks are determined using a built-in mathematical model's genetic algorithm (GA) and a neural network algorithm.

13. The method of claim 1, wherein isolating the EV samples from the first tear fluid or second tear fluid comprises:
eluting the first tear fluid or second tear fluid to form a first tear fluid mixture or a second tear fluid mixture;
centrifuging the first tear fluid mixture or the second tear fluid mixture to collect a first supernatant or a second supernatant;
centrifuging the first supernatant or the second supernatant to remove cells and impurities;
filtering the first supernatant or the second supernatant through a nanosized filter to remove debris, thereby collecting a first intermediate or a second intermediate; and
isolating the EV samples from the first intermediate or the second intermediate in a microfluidic device.

14. The method of claim 1, wherein the proteomes expression of the EV samples isolated from the first tear fluid is characterized by Silver Staining and Western Blot.
